(19) United States
(12) Patent Application Publication
(10) Pub. No.: US 2023/0018851 A1
(43) Pub. Date: Jan. 19, 2023

(54) USE OF HYALURONIC ACID DERIVATIVES IN THE REGENERATION OF BONE AND CARTILAGE TISSUES

(71) Applicant: TRB CHEMEDICA INTERNATIONAL SA, Geneva (CH)

(72) Inventors: Demetrio MANENTI, Paradiso (CH); Giuliana ABBIATI, Paradiso (CH)

(21) Appl. No.: 17/786,752

(22) PCT Filed: Dec. 18, 2020
(86) PCT No.: PCT/IB2020/062186

§ 371 Date: Jun. 17, 2022

(30) Foreign Application Priority Data
Dec. 20, 2019 (IT) 102019000025126

(51) Int. Cl. A61K 31/198; A61K 31/728; A61P 19/04; A61K 31/505

(52) U.S. Cl. CPC A61K 31/198; A61K 31/728; A61P 19/04; A61K 31/505

(57) ABSTRACT

The present invention relates to the use of derivatives between hyaluronic acid, heterocyclic compounds and naturally occurring amino acids in single, oligomeric or polymeric form for the treatment of skeletal diseases, in particular in the regeneration of bone and cartilage tissues.

The present invention relates to the use of derivatives between hyaluronic acid, heterocyclic compounds and naturally occurring amino acids in single, oligomeric or polymeric form for the treatment of skeletal diseases, in particular in the regeneration of bone and cartilage tissues.

### STATE OF THE ART

In the biological field, a tissue is defined as a set of cells, structurally similar, associated by function. It therefore constitutes a higher level of cellular organization, with a specific role to play within an organism.

With regards to the animal kingdom, including humans, four fundamental types of tissues can be recognized: epithelial, connective, muscular and nervous tissue, in turn divided into more specialized subtypes. In higher animals, different tissues combine to form further organized structures: the organs.

Connective tissues, such as bone, adipose, fibrous and trophic tissues, are tissues consisting of separate cells between which non-living material, called extracellular matrix (ECM), is interposed. This matrix can be liquid or rigid; two extreme examples are blood, in which the matrix is the plasma, and bones in which there is a mineralized tissue and an extremely rigid matrix. Precisely because of this peculiar characteristic, sometimes bone tissue is referred to as “hard tissue”; in contrast to “soft tissues” which generally refer to the other connective tissues.

Cartilage, the precursor tissue of bone, is also part of the connective tissues. It consists mainly of cells called chondrocytes that are able to produce a high amount of extracellular matrix mainly composed of collagen, elastin and proteoglycans.

Tissue regeneration, which must take place after injuries or diseases in order to ensure complete healing, is a process based on the renewal and differentiation of the cells of the tissue(s) involved.

Regenerative medicine is an emerging field of research that has gained interest in recent years; it combines different aspects of medicine, cell biology and bioengineering with the ultimate goal of regenerating, repairing or replacing the damaged or missing tissues. The different lines of research, involving both differentiated cells and stem cells, aim to optimize the regeneration, healing and/or replacement of the damaged tissues.

One of the most widely used approaches in the field of bone and cartilage tissue regeneration today is based on a mesenchymal stem cell (MSC) approach.

As far as cartilage is concerned, although the differentiation of cartilage from MSCs has been demonstrated, the clinical use of this approach is still limited and not completely successful. Cartilage regeneration can also be achieved by methods such as tissue grafts (autografts or allografts) or by techniques known in literature adapted to stimulate the natural repair process. The techniques identified to date as the most reliable in the case of cartilaginous tissues therefore aim either to improve the regenerative properties of the tissues or consist of transplantation of chondrocytes with the aim of increasing or healing the residual tissue.

As far as bone tissue is concerned, marrow is still the source of choice for isolating MSCs from which to obtain differentiated bone cells, but other sources for obtaining MSCs such as dental tissue are also known.

Finally, it is known that the extracellular matrix plays an important role in the cellular differentiation of connective tissues; in particular the interaction of MSCs with the ECM can improve the osteogenic differentiation of these cells. In fact, the ECM contains a variety of macromolecules, including collagen, adhesive glycoproteins and glycosaminoglycans (GAGs), which not only has a role of supporting cells and determining tissue structure, but also contribute to the spread of growth factors and to the cellular interactions with the micro-environment, thus influencing the cell behavior.

Despite numerous studies and progress made in the recent years in the context of use of stem cells, the reconstruction of bone and cartilage tissue defects is still a challenge for the regenerative medicine, as current known treatments are only partially effective and not always feasible.

There is therefore the need to find new ways and approaches to deal more easily and effectively with the problem of regeneration and healing of the bone and cartilage tissues of the organism.

### DESCRIPTION OF THE INVENTION

A class of hyaluronic acid derivatives, in which this molecule is associated with at least one heterocyclic compound derived from a purine or pyrimidine base and with at least another organic compound consisting of a naturally occurring amino acid, in single, oligomeric or polymeric form, is described in EP1525244 with the procedures for its preparation.

The above mentioned derivatives of hyaluronic acid, a glycosaminoglycan (GAG) naturally present in the extracellular matrix (ECM), constitute a more stable form than the native one because the compounds associated with it are found in the target sites of the lytic hyaluronidase enzyme, normally responsible for its degradation, making its action more difficult. Furthermore, depending on the type of heterocyclic compounds and amino acids selected, these derivatives show a particular three-dimensional structure, which makes them able to modify the micro-environment of the EMC.

Object of the present invention is therefore the use of said derivatives of the hyaluronic acid in the regeneration of the bone and cartilage tissues, thanks to the high regenerative potential on said tissues highlighted in the experimental section accompanying the present description.

The compounds of hyaluronic acid and at least one heterocycle derived from purine and/or pyrimidine, associated with at least one different organic compound selected from the naturally occurring amino acids in the single, oligomeric or polymeric form, thus are used, according to the present invention, in the treatment of the skeletal conditions, in particular in the regeneration of hard tissues.

According to the present invention, the hyaluronic acid derivatives the use of which is object of the present invention, are high molecular weight hyaluronic acid, in the range between 400,000 and 4 million Da, preferably between 800,000 and 3.5 million Da, more preferably between 1.5 and 3 million Da.

Optionally, the hyaluronic acid derivatives the use of which is object of the present invention, consist of low molecular weight hyaluronic acid, for example, in the range between 80,000 and 400,000 Da.

The molecular weights of the polymers in general and hyaluronic acid in particular are, for example, the number average molecular weight Mₙ defined as the average of the weights of polymer chains:

Mₙ=Σ₍ᵢ₎NᵢMᵢ/Σ₍ᵢ₎Nᵢ

wherein Mᵢ is the molecular weight and Nᵢ is the number of chains or the weight average molecular weight M_{w} which is defined as:

M_{w}=Σ₍ᵢ₎NᵢMᵢ²/Σ₍ᵢ₎NᵢMᵢ

This quantity is more influenced by the fraction with higher molecular weights and is higher than the weight average molecular weight.

The determination of the average molecular weight of a polymer is of great importance as it represents the primary characteristic of the polymer to which many of its properties are related. The molecular weight can be obtained by various techniques including the centrifugation technique (sedimentation balance), light scattering technique and osmometry.

The speed at which molecules settle in an ultracentrifuge is proportional to their molecular weight: assuming, in fact, that the molecular weight increases as its volume increases, the molecular weight can be determined based on the speed of sedimentation.

The light scattering technique is based on the principle that when a light beam crosses an empty space in a straight line, it does not lose energy on its path. If, on the other hand, particles of any kind are present in the space, it is possible to observe that the light beam scatters or is subjected to deviations in all directions by the particles present. The primary light beam thus loses some of its energy and decreases in intensity.

It is possible to develop a theory for the determination of the molecular mass of an M polymer by measuring the intensity of the scattered light, and therefore the photo-diffusivity of the polymer itself placed in a diluted solution in a suitable solvent.

The molecular mass obtained has an average value and it can be demonstrated that in this case it is the weight average.

Of the above mentioned methods, the most important and widespread is osmometry. The value π of the osmotic pressure for solutions with different polymer concentration C is measured. Remembering that

π=CRT

When the temperature T at which the measurement is carried out is known, the molecular weight value will be calculated, remembering that C=mass/molecular weight.

The most widely used standard technique for the determination of the chemical-physical characteristics of a polymer is instead called GPC (Gel Permeation Chromatography).

According to the present invention, the heterocyclic compounds selected are derivatives of purine bases selected for example from adenine and guanine, and/or pyrimidine compounds selected for example from thymine, cytosine and uracil. The preferred base, according to the invention, is a pyrimidine base such as thymine. Other purine or pyrimidine derivatives which can be used to form compounds the use of which is object of the present invention, may be selected from: 5,6-di-hydrouracil, 1-methyluracyl, 3-methyluracyl, 5-hydroxymethyluracyl, 2-thiouracil, N⁴-acetyl cytosine, 3-methylcytosine, 5-methylcytosine, 5-hydroxymethylcytosine, 1-methyladenine, 2-methyladenine, 7-methyladenine, N⁶-methyladenine, N⁶,N⁶-dimethyladenine, N⁶-(Δ²-isopentenyl)adenine, 1-methylguanine, 7-methylguanine, N²-methylguanine, N²,N²-dimethylguanine.

Preferably, in the derivatives of hyaluronic acid the use of which is object of the present invention, the interaction between the hyaluronic acid chain and the purine or pyrimidine bases takes place thanks to at least one ionic bond between a —COOH residue of said acid and a basic center, in particular a basic nitrogen, of said heterocyclic bases.

In practice, according to the present invention, hyaluronic acid is reacted with at least one purine and/or pyrimidine base selected from those set forth above, under reaction conditions allowing the formation of at least one type of ionic bond between at least one acid center of the hyaluronic acid, such as for example a free carboxyl group in acid or carboxylate salt form, and at least one basic center of the purine and/or pyrimidine base, also in free base or ammonium salt form.

According to the present invention, the hyaluronic acid derivatives the use of which is object of the present invention may contain more than one type of purine and/or pyrimidine base with a variable reciprocal ratio; said derivatives may therefore be represented by “mixed” salts consisting of a variable number of purine/pyrimidine bases.

The hyaluronic acid derivatives, the use of which is object of the present invention, also include at least one naturally occurring amino acid, or an oligomer or polymer thereof, to provide an additional salification product thanks to the —COOH groups present and remaining free in the structure of the hyaluronic acid. The amino acids that can be used to form these derivatives are for example selected from: alanine, arginine, asparagine, aspartic acid, glutamic acid, cysteine, phenylalanine, glutamine, glycine, histidine, isoleucine, leucine, lysine, methionine, proline, serine, tyrosine, threonine, tryptophan and valine. Preferably said amino acids are selected from lysine and alanine.

The characteristics of said derivatives reflect those of hyaluronic acid, purine and/or pyrimidine bases and amino acids which, in turn, are bound by at least one easily hydrolyzable ionic-type bond, making the different components easily accessible in situ.

Particularly preferred is the compound currently marketed under the name T-LysYal® (T-Lys), a derivative of hyaluronic acid, lysine and thymine by the formation of ionic-type bonds.

According to the present invention, said compounds between hyaluronic acid, at least one heterocyclic compound selected from a purine and/or pyrimidine derivative and at least one naturally occurring amino acid, or its oligomer or polymer, can be advantageously used to induce and stimulate cell differentiation in the osteogenic and chondrogenic lineage of MSCs.

According to the present invention, it is therefore possible to use the hyaluronic acid derivatives described above for the treatment of skeletal conditions, in particular in the regeneration of bone and cartilage tissues.

Still according to the present invention, said hyaluronic acid derivatives are advantageously used, as already mentioned, to induce and stimulate cell differentiation in the osteogenic and chondrogenic lineage of MSCs.

According to the invention, it is also possible to integrate said hyaluronic acid derivatives into suitable pharmaceutical formulations and/or implantable scaffolds, which can be used to support tissue regeneration of bones and cartilages.

Said derivatives can further be used in therapy for the repair and regeneration of bone and cartilage tissues.

It is an object of the present invention the use of said derivatives in the treatment related to the tissue regeneration of bone and cartilage tissues. According to the present invention, said derivatives can be advantageously integrated into implantable scaffold systems or other pharmaceutically appropriate formulations.

By other “pharmaceutical appropriate formulations” it is meant, but not limited to, solutions and/or suspensions for parenteral use, solid forms (e.g. tablets, capsules, granules) or semi-solid forms (e.g. gels, pastes, creams, ointments) for oral or topical use, intramuscular and/or subcutaneous implants and other formulations known to the expert in the art.

The potential of the invention will now be described in the Experimental Section below, wherein studies carried out by using said hyaluronic acid derivatives are set forth, in relation to their potential for cellular regeneration of the hard tissues of the organism. The following examples are intended for illustrative purposes only and in no way by limitation.

### BRIEF DESCRIPTION OF THE FIGURES

FIG. 1: Effect of T-Lys in the differentiation of MSCs towards the osteoblastic lineage. Sections: A) qPCR carried out on DBSCs grown with osteogenic medium and stimulated with 0.3% T-Lys and control DBSC; B) immunoblotting test for the expression of the Runx-2 and Col 1 proteins; C) histochemical assay on the ALP enzyme (purple staining).

FIG. 2: Effect of T-Lys in the deposition of mineral matrix during the osteogenic differentiation of MSCs. Deposition of mineral matrix tested by ARS (red staining) in cells treated with T-Lys, hyaluronic acid and control.

FIG. 3: Effect of T-Lys on the expression of typical markers in chondrocyte cultures. qPCR carried out on chondrocyte pellet cultures grown with chondrogenic medium and stimulated with 0.3% T-Lys and negative control group (Ctr).

FIG. 4: Effect of T-Lys on the chondrocyte proliferation and tissue growth. Sections: A) images and measurements of culture pellets of dissected chondrocyte treated with T-Lys and control groups; B) images and measurements of the deposition of cartilage matrix of dissected chondrocytes treated with T-Lys and control groups; C) theoretical reconstruction of the thickness of the culture pellets of chondrocytes treated with T-Lys and control groups.

### EXPERIMENTAL SECTION

### Example 1

Effect of T-Lys in the Differentiation of MSCs Towards the Osteoblastic Lineage

The stem cells of the dental germ (DBSC) were used as a source of MSC and were differentiated for 12 days in osteogenic medium. A portion of the tested cells was treated with 0.3% T-Lys (T-Lys—treatment group) added to the culture medium with each change. The fraction of cells not treated with T-Lys was used as control group (Ctr). The mRNA levels of the early markers of typical osteoblasts, Runx-2 and Collagen I (Col 1) were determined in Ctr and T-Lys samples by using real time PCR.

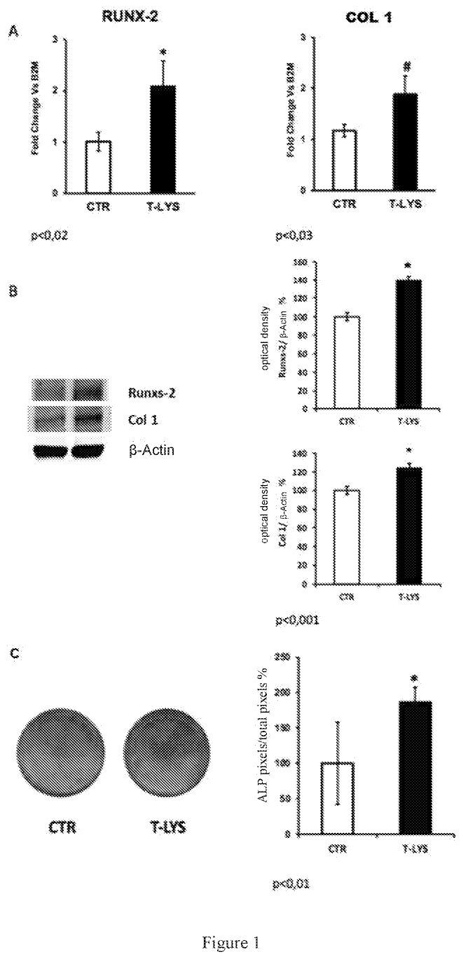

FIG. 1(A) shows how the expression of both markers has increased significantly in T-Lys-treated cells compared to Ctr cells, suggesting that T-Lys treatment has improved the ability of MSCs to differentiate in the lineage of the osteoblasts.

The protein expression levels of these osteoblastic markers were further evaluated in T-Lys and Ctr cells by Western Blot analysis.

FIG. 1(B) highlights how the level of the Runx-2 and Col 1 proteins is increased in T-Lys-treated cells compared to Ctr cells, thus confirming the mRNA expression trend.

A histochemical test was then carried out to explore the expression of another marker of osteoblastic cells, the alkaline phosphatase (ALP) enzyme, in response to the treatment with T-Lys. The result of this experiment, shown in FIG. 1(C), revealed that the stimulation of MSCs with T-Lys, during osteogenic differentiation, significantly increased the purple staining that identifies ALP expression.

All the results described above have demonstrated that T-Lys is able to increase the ability of MSCs to differentiate into cells similar to osteoblasts.

### Example 2

Effect of T-Lys in the Deposition of Mineral Matrix During the Osteogenic Differentiation of MSCs

In order to thoroughly investigate the effect of this new molecule on the osteogenic differentiation of MSCs, DBSC culture under mineralization conditions was followed for 21 days on different samples: Ctr (without any addition as negative control group); HA (with the addition of unmodified hyaluronic acid as positive control group) and T-Lys (in which cells were treated with 0.3% T-Lys as treatment group).

The effect of T-Lys on the deposition of DBSC mineral matrix was analyzed using the Alizarin Red Staining (ARS) histochemical test quantified by using a colorimetric technique. The mineralization capacity of the cells treated with 0.3% T-Lys demonstrated to be significantly higher than both Ctr and HA.

These data show how T-Lys is able to increase the osteogenic capacity of MSCs by also stimulating their ability to produce mineralized matrix.

### Example 3

Effect of T-Lys on the Influence of the Sub-Cellular Distribution of Δvβ3 Integrin

Integrins are receptors for ECM molecules, important in cell adhesion but also in the mediation of proliferation and differentiation signals. In particular, the αvβ3 integrin is the receptor for the bone protein called Osteopontin, of fundamental importance to determine the differentiation of MSCs towards the osteogenic lineage. Therefore, it was assessed whether the treatment with T-Lys could influence the subcellular distribution of αvβ3 integrin.

The subcellular distribution of said integrin was analyzed by confocal microscopy in DBSCs treated with T-Lys and Ctr. The analyses were carried out, after only 4 days of osteogenic differentiation, to compensate for the fact that the cells show a rapid propensity to form a multilayer that prevents their microscopic observation. In Ctr cells the αvβ3 integrin proved to be distributed in several sites, while the T-Lys treatment induced a different organization of this receptor, more localized in the focal adhesion sites. Therefore, after 4 days of differentiation, the receptor under controlled conditions was still distributed throughout the cell, while in T-Lys cells it was present in the focal adhesions. The presence of “strings” (the typical pattern of αvβ3 integrins involved in focal adhesions) was detectable in T-Lys cells and not in Ctr cells. These results suggest that the effect of T-Lys on DBSC differentiation could be mediated by αvβ3 rearrangement.

### Example 4

Effect of T-Lys on the Expression of the Typical Markers in Chondrocyte Cultures

Human joint chondrocytes collected from patients undergoing orthopedic surgery have been grown in pellet cultures in order to mimic the micro-architecture of the three-dimensional tissue and avoid the improper de-differentiation of chondrocytes that easily occurs when they are grown in two dimensions. The cell pellets were grown for 28 days under chondrogenic conditions. The control group (Ctr) was treated according to the conventional protocol while the T-Lys group was added with 0.3% T-Lys, at each change of vehicle. At the end of the culture period, the chondrocyte culture pellets were lysed and evaluated for the gene expression analysis. The mRNA levels of typical chondrogenic markers: Sox-9, Collagen II (Col II), Collagen X (Col X) and Aggrecan were determined in both groups of real time PCR samples.

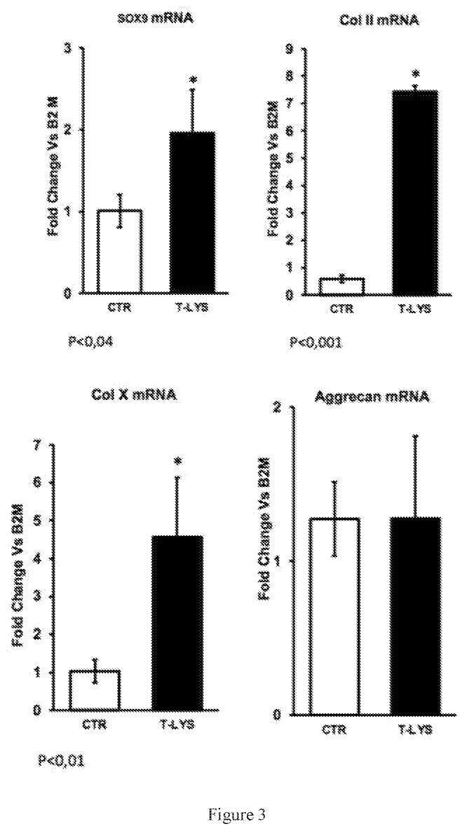

FIG. 3 shows the results of these tests which demonstrate that, for three out of these four markers taken into account, their expression has definitely increased in the T-Lys groups. In particular, the expression of Sox-9, which is the main transcription factor involved in the chondrogenic differentiation, is significantly increased in T-Lys-treated cells, compared to Ctr cells. In accordance with this result, Col II and Col X, the proteins typical of the extracellular matrix of the cartilage, are also increased thanks to the treatment with T-Lys, indicating once again that this molecule supports and improves the differentiation of chondrocytes. On the other hand, the expression of Aggrecan, a proteoglycan of the cartilage, was not affected in any way.

### Example 5

Effect of T-Lys on the Chondrocyte Proliferation and Tissue Growth

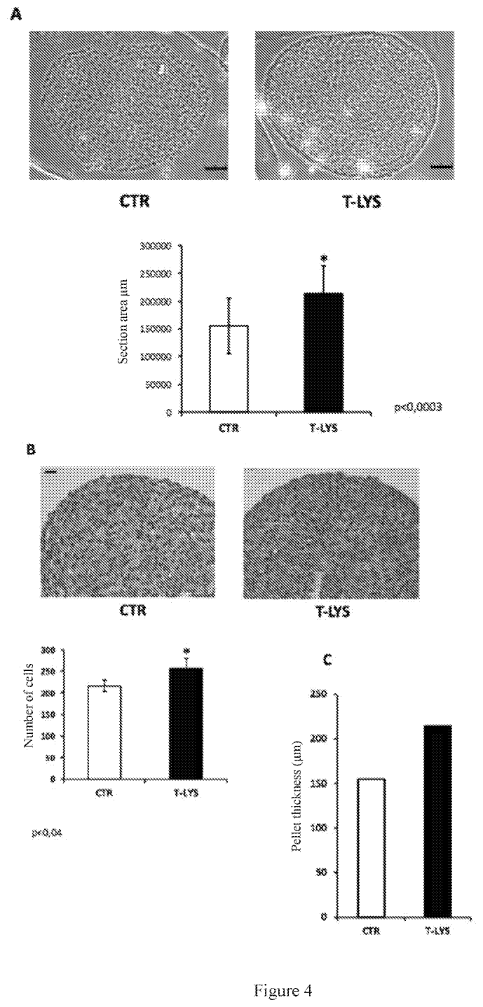

After 28 days of differentiation under the conditions described in Example 4, the chondrocyte pellets were fixed with 4% paraformaldehyde, and were incorporated, sectioned and histologically stained and examined. The morphometric examination by optical microscope of culture pellets of the dissected chondrocytes revealed that the T-Lys ones were larger than those in the control group. This result is visible in FIG. 4(A).

To quantify the pellet size, the samples were sectioned (5 μm thickness) and the area of each section, obtained for the two groups of cells, was measured by ImageJ software. The graph in FIG. 4(A) shows that the average surface area was significantly larger in the T-Lys-treated group than in the control group.

The pellets were then stained with Safranin O to highlight the chondrocytes (FIG. 4(B)). The staining revealed the presence of cartilaginous matrix (orange staining), demonstrating that the cells were able to differentiate and produce CME components under these culture conditions; the nuclei were counter stained with hematoxylin (FIG. 4(B)). To check whether the T-Lys treatment also had an influence on the number of cells, the cells in a selected field (100×100 μm) were counted for each section. The graph in FIG. 4(B) depicts the number of cells for the two cultures and it can be seen that it has significantly increased in the case of the treated group compared to the control group. It is interesting to note that it was possible to obtain a larger number of sections with uniform thickness (5 μm), called “slices”, in the case of the T-Lys sample compared to the control group.

This difference is depicted in the graph of FIG. 4(C), in which the number of “slices” has been multiplied by the thickness of the section (5 μm) thus reconstructing a theoretical thickness of the culture pellet as a whole. These results demonstrate that T-Lys stimulates the proliferation of chondrocytes, their differentiation and matrix secretion.

### DETAILED DESCRIPTION OF THE FIGURES

FIG. 1: Effect of T-Lys in the differentiation of MSCs towards the osteoblastic lineage

A) qPCR carried out on DBSCs grown with osteogenic medium and stimulated with 0.3% T-Lys and DBSC Ctr. Each graph depicts the average±standard error of 3 independent experiments performed in triplicate. *P<0.02 compared to the control group. The expression has been normalized to mic2microglobulin (B2M). The graphs show that the treatment with T-Lys significantly increased the expression of the two osteoblast markers Runx-2 and Coll.

B) Immunoblotting test for the expression of the proteins Runx-2 and Col 1; each graph depicts the average optical density calculated in relation to a constituent protein (0-Actin housekeeping gene)+standard error of 3 independent experiments carried out in triplicate. *P<0.001 compared to the control group. Representative immunoblot images were also depicted on the left side of the figure. The graphs show how the measured parameter is higher in T-Lys-treated cells than in the control group.

C) Histochemical assay on the ALP enzyme (purple staining) carried out on DBSCs maintained under osteogenic conditions for 7 days and stimulated with T-Lys compared to the control group. The graph depicts the quantification of positive staining in percentage with respect to the control group (*P<0.01) and derives from the analyses of 3 independent experiments carried out in quadruplicate. The data are shown as an average±standard error. Representative images of culture wells are also depicted on the left in the figure. The graph shows how T-Lys samples have a higher expression of the alkaline phosphatase enzyme.

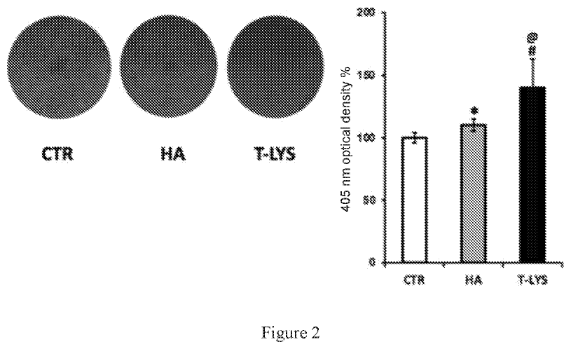

FIG. 2: Effect of T-Lys in the deposition of mineral matrix during the osteogenic differentiation of MSCs

Deposition of mineral matrix tested by ARS (red staining) in cells treated with T-Lys, hyaluronic acid and Ctr under osteogenic conditions for 21 days. The graph shows the quantification of optical density of the dye extracted from the colored cell layers as average percentage±standard error and is representative of 3 independent experiments carried out in quadruplicates. *P<0.01, #P<0.001 versus negative control group (Ctr); @P<0.01 versus positive control group (HA). Representative images of culture wells are also depicted on the left in the figure. The graph shows how T-Lys samples have a higher deposition of mineral matrix than both the untreated sample and the sample treated with native hyaluronic acid.

FIG. 3: Effect of T-Lys on the expression of the typical markers in chondrocyte cultures

qPCR carried out on chondrocyte pellet cultures grown with chondrogenic medium and stimulated with 0.3% T-Lys and negative control group (Ctr). Each graph depicts the average±standard error of 3 independent experiments performed in triplicate. *P<0.04 for Sox-9, *P<0.001 for Col II, *P<0.01 for Col X compared to the control group. The expression has been normalized to mic2microglobulin (B2M). The graphs show that treatment with T-Lys significantly increased the expression of Sox-9, Col II and Col X chondrocyte markers, while it had no effect on the Aggrecan expression.

FIG. 4: Effect of T-Lys on the chondrocyte proliferation and tissue growth

A) The culture pellets of sectioned chondrocytes were photographed under an optical microscope by using a 20× objective lens and analyzed by using the Image-J software for morphometric examination of the areas. The selected images are representative of three different experiments, scale bar depicted in the bottom right corner of the figures: 75 μm. The graph depicts the average±standard error of 3 independent experiments performed in triplicate, *P<0.0003. The pellets treated with T-Lys appear larger than those in the control group.

B) The cartilage matrix deposition was measured by using Safranin O staining and the chondrocyte nuclei were counter stained with hematoxylin. The images were taken with a 40× lens, scale bar shown in the upper left corner of the control figure: 25 μm. The graph depicts the average±standard error of 3 independent experiments performed in triplicate, *P<0.04. The number of cells in the T-Lys sample is greater than the number in the control group.

C) The graph shows a theoretical reconstruction of the thickness of the chondrocyte culture pellet in which the number of sections obtained was multiplied by the thickness of the slice and expressed in μm. The group treated with T-Lys shows a greater thickness.

1. A compound of hyaluronic acid and at least one heterocycle derived from purine and/or pyrimidine, said compound being further associated with at least one different organic compound selected from naturally occurring amino acids in the single, oligomeric or polymeric form, for its use in the treatment of skeletal conditions, in particular in the regeneration of hard tissues.

2. The compound for the use according to claim 1, wherein said at least one heterocycle is thymine.

3. The compound for the use according to claim 1 or 2, wherein said at least one different organic compound is lysine.

4. The compound for the use according to anyone of the preceding claims, wherein the bond between said hyaluronic acid, said heterocycle and said naturally occurring amino acid is a chemical bond of ionic type.

5. The compound for the use according to anyone of the preceding claims, wherein said compound of the hyaluronic acid is T-LysYal®, which comprises hyaluronic acid in combination with lysine and thymine.

6. The compound according to anyone of the preceding claims, for its use in the induction and stimulation of cell differentiation in the osteogenic and chondrogenic lineage of mesenchymal stem cells.

7. The compound for its use according to claim 6, wherein said mesenchymal stem cells are stem cells of the dental germ.

8. An implantable scaffold comprising a compound of hyaluronic acid and at least one heterocycle derived from purine and/or pyrimidine, said compound being further associated with at least one different organic compound selected from naturally occurring amino acids in the single, oligomeric or polymeric form.

9. The implantable scaffold according to claim 8, for its use in the treatment of skeletal conditions, in particular in the regeneration of hard tissues.

10. A pharmaceutical formulation comprising a compound of hyaluronic acid and at least one heterocycle derived from purine and/or pyrimidine, said compound being further associated with at least one different organic compound selected from naturally occurring amino acids in the single, oligomeric or polymeric form, for its use in the treatment of skeletal conditions, in particular in the regeneration of hard tissues.
